(19) United States
(12) Patent Application Publication
(10) Pub. No.: US 2011/0020936 A1
(43) Pub. Date: Jan. 27, 2011

(54) METHOD FOR ELECTROPORATION OF LACTOBACILLUS BUCHNERI WITH NUCLEIC ACID

(75) Inventor: Brenda K. Smiley, Granger, IA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignee: Pioneer Hi-Bred International, Inc., Johnston, IA (US)

(21) Appl. No.: 12/843,327

(22) Filed: Jul. 26, 2010

Related U.S. Application Data
(60) Provisional application No. 61/228,375, filed on Jul. 24, 2009.

(51) Int. Cl. C12N 15/74

(52) U.S. Cl. 435/471

(57) ABSTRACT

Electroporation methods for transferring a nucleic acid of interest, including ferulic acid esterase nucleic acid, into bacterial host cells, such as Lactobacillus spp. are disclosed. Compositions of transformed bacterial host cells, such as transformed Lactobacillus buchneri, and use assays are provided.

### CROSS-REFERENCE TO RELATED APPLICATIONS

This application claims the benefit of U.S. Provisional Patent Application No. 61/228,375, filed Jul. 24, 2009, which is hereby incorporated herein in its entirety by reference.

### FIELD OF THE INVENTION

The field of the disclosure relates generally to bacterial molecular biology. More specifically, the disclosure relates to the use of electroporation to transform Lactobacillus species (Lactobacillus spp.) into specific strains by introduction of nucleic acid.

### BACKGROUND OF THE INVENTION

Lactobacillus spp. play large roles in the food and agriculture industries, as many members of the species express enzymes capable of breaking down plant cells walls. For example, selected native Lactobacillus buchneri strains produce a ferulate esterase enzyme capable of enhancing fiber digestion in animals. Unfortunately, due to the lack of efficient transfer, experimentation on modifying Lactobacillus enzymes through nucleic acid transformation has been limited, as transformation of certain Lactobacillus spp. into more beneficial strains often requires excessive experimentation.

Electroporation has been used to increase the efficiency of transfer of nucleic acid into prokaryotic cells. Nevertheless, this is not a smooth process as use of electroporation for transformation through nucleic acid transfer depends on a variety of factors, including, but not limited to, electrical field strength, pulse decay time, pulse shape, temperature in which the electroporation is conducted, type of cell, type of suspension and washing medias, mixing conditions, and the concentration and size of the nucleic acid to be transferred.

### BRIEF SUMMARY OF THE INVENTION

Methods are provided for transferring a nucleic acid of interest into Lactobacillus spp. cells. The methods involve culturing the Lactobacillus spp. cells, such as, for example, Lactobacillus buchneri cells, in a growth medium. In certain embodiments of the invention the Lactobacillus spp are cultured in a growth medium comprising an agent to weaken the cell wall. The methods further involve harvesting and washing the Lactobacillus spp. cells, followed by mixing the Lactobacillus spp. cells with at least one nucleic acid of interest and then subjecting the Lactobacillus spp. cells plus nucleic acid of interest to electroporation, thereby permitting the transfer of the nucleic acid of interest into the Lactobacillus spp. cells.

In one embodiment, a method for transferring a nucleic acid of interest into a bacterial host cell is provided. In many cases, the bacterial host cell will be an obligately heterofermentative Lactobacillus sp. For example, the obligately heterofermentative Lactobacillus sp. can be Lactobacillus buchneri. The bacterial host cells are cultured in a growth media that contains an agent to weaken the cell wall. Following culture, the bacterial host cells are harvested, washed, and mixed with a nucleic acid of interest. The nucleic acid of interest is transferred into the host cell using electroporation. General electroporation parameters include a field strength of between about 0.5 kV/cm and 25 kV/cm and a time constant of between about 2.5 milliseconds to 25 milliseconds.

Methods are provided for constructing a new strain of Lactobacillus buchneri. In constructing this strain, the nucleic acid of interest will be a mutant nucleic acid of interest, such as a knock-out. This mutant nucleic acid of interest is then transferred into Lactobacillus buchneri cells using electroporation, where the general electroporation parameters include a field strength of between about 0.5 kV/cm and 25 kV/cm and a time constant of between about 2.5 milliseconds to 25 milliseconds. Also provided is a Lactobacillus buchneri strain is prepared by the methods of present invention.

Further provided is an assay for studying the impact of ferulic acid esterase activity is described. Lactobacillus sp. strains containing mutant ferulic acid esterase, such strains made using the methods of the disclosure are added to whole plant silage, as are native Lactobacillus sp. The difference in digestibility of the whole plant silage is then measured as between the plant silage with a mutant ferulic acid esterase and that with a native ferulic acid esterase.

Also provided are Lactobacillus spp. cells comprising a nucleic acid of interest that was transferred into the Lactobacillus spp. cells by the methods of the present invention. In one embodiment, Lactobacillus spp. cells comprise a non-functional ferulic acid esterase gene that was transferred into the Lactobacillus spp. cells by the methods of the present invention.

### DETAILED DESCRIPTION OF THE INVENTION

The present invention now will be described more fully hereinafter. The invention may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the invention set forth herein will come to mind to one skilled in the art to which the inventions pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes, and symbols may be denoted in their SI accepted form. Numeric ranges recited within the specification are inclusive of the numbers defining the range and include each integer within the defined range.

Bacterial cells that are subjected to electroporation to have a nucleic acid of interest transferred into them are known as bacterial host cells. Embodiments include methods for transferring a nucleic acid of interest into bacterial host cells that are Lactobacillus spp. In certain embodiments, the Lactobacillus sp. will be in the group of obligately heterofermentative Lactobacillus. Individual obligately heterofermentative Lactobacillus include, but are not limited to, Lactobacillus brevis, Lactobacillus buchneri, Lactobacillus fermentum, and Lactobacillus reuteri. Lactobacillus Molecular Biology: From Genomics to Probiotics, Asa Ljungh and Torkel Wadstrom, eds., hereby incorporated by reference, provides a review of the different obligately heterofermentative Lactobacillus spp. anticipated to be used with many of the current embodiments.

A nucleic acid of interest can be transferred into bacterial host cells capable of producing ferulate esterase. Ferulic acid esterase and ferulate esterase are interchangeably used herein. Ferulate esterase producing bacteria include Lactobacillus strains. In one embodiment, the ferulate esterase producing Lactobacillus is Lactobacillus buchneri. In an additional embodiment, the ferulate esterase producing Lactobacillus sp. is Lactobacillus brevis. Other ferulate esterase producing bacteria that may have nucleic acids of interest transferred into them include Lactobacillus plantarum, Lactobacillus sanfranciscensis, Lactobacillus reuteri, Lactobacillus alimentarius, Lactobacillus crispatus, and Lactobacillus paralimentarius. As one skilled in the art understands, bacterial species capable of producing ferulate esterase need not be native species but can be functional mutations of native species or mixtures thereof. As used herein, “functional mutations” mean mutations that have been directly or indirectly obtained by genetic modification of, or using, the native nucleic acid. Such mutation retains at least 50% of the activity of the native nucleic acid. The genetic modification can be achieved through any means, such as but not limited to, chemical mutagens, ionizing radiation, transposon-based mutagenesis, or via conjugation, transduction, or transformation using the referenced strains as either the recipient or donor of genetic material. Use of several different strains of each of the species of Lactobacillus cells is contemplated. For example, a Lactobacillus buchneri strain can be strain PTA 6138. In a different embodiment, the strain includes but is not limited to PTA-2493, PTA-2494, PTA-2495, ATCC202118, NRRL B-30986, NRRL B-30987, NRRL B-30988, NRRL B-30989, NRRL B-30990, NRRL B-30991, or NRRL B-30866.

An initial step, prior to transferring a nucleic acid of interest into a bacterial host cell, includes culture of bacterial host cells in an appropriate culture medium. As used herein, a culture medium is a liquid or gel designed to support the growth of bacterial cells. In many embodiments, the growth media will specifically support the growth of Lactobacillus spp. An example of such a growth media is De Man, Rogosa, Sharpe (MRS) broth. MRS broth can be purchased commercially or made in the laboratory from its individual components. Generally, the formula for MRS broth includes: 1.0% peptone, 1.0% meat extract, 0.5% yeast extract, 2.0% glucose, 0.5% sodium acetate trihydrate, 0.1% polysorbate 80, 0.2% ammonium citrate, 0.2% dipotassium phosphate, 0.01% magnesium sulfate heptahydrate, and 0.005% manganese sulfate tetrahydrate. Nevertheless, the skilled artisan understands that this formula is approximate only and that any specific composition of MRS broth can be altered slightly. The MRS broth may also be pH adjusted.

A culture medium can be supplemented in exemplary embodiments with additional agents, such as agents that weaken the bacterial host cell wall and are osmotic stabilizers. Agents to weaken the bacterial host cell wall include, but are not limited to, threonine, lysozyme, glycine, penicillin, detergents, and bacitracin. As the skilled artisan understands, “weakening the bacterial host cell wall” and “increasing the permeability of the bacterial host cell wall” are interchangeable and any agent that increases the permeability of the bacterial host cell wall without killing a majority of the bacterial host cells can be used. Concentrations of bacterial host cell wall permeabilization agents largely depend on the type of agent being used and will vary with different embodiments. For example, some embodiments may use a glycine as the bacterial host cell wall weakening agent. The glycine concentration in these embodiments can be between about 0.1% (w/v) and 5% (w/v), wherein at least one of those embodiments uses a concentration of glycine of about 1% (w/v).

Osmotic stabilizers are well known in the art. An osmotic stabilizer is an agent that functions to stabilize the bacterial host cell membrane against osmotic pressure and increase the efficiency of biochemical reactions in the membrane. These stabilizers include sucrose, sorbitol, mannitol, potassium chloride, and magnesium sulfate. Usually the concentration of these stabilizers varies between about 0.1 M and 1.5 M. In some embodiments, sucrose will be used as an osmotic stabilizer. The sucrose concentration can be between about 0.1 M and about 1.0 M, preferably about 0.5 M. In other embodiments, it is preferable to use magnesium sulfate.

The bacterial host cells in some embodiments will be cultured until the culture is in the logarithmic phase of growth. The term logarithmic phase means the stage of bacterial host cell growth when the logarithmic correlation of the number of bacterial host cells or the turbidity against the cultivation period becomes linear, i.e. namely the stage when the growth of the bacterial host cells becomes most active after an initial lag phase when almost no growth is observed. Bacterial growth has long been studied by microbiologists. Therefore, one with skill in the art will easily be able to measure and follow bacterial host cell growth phases. The methods of determining the phase of bacterial host cell growth are not meant to be limiting and encompass, among others, measurement through microscopic methods, flow cytometry, colony counting, turbidity, nutrient uptake and other such methods known in the art. In selected embodiments, the growth phase of the bacterial host cells will be measured using turbidity. Turbidity can be measured using various methods. In one embodiment, an electronic meter, such as a spectrophotometer will be used to measure the turbidity. In other embodiments, turbidity will be measured with a turbidimeter. Skilled artisans can easily determine the particular turbidity that goes with a specific phase of growth.

In some embodiments, the bacterial host cells will be cultured until they are in the early logarithmic phase of growth. As used herein, early logarithmic phase of growth will be growth in the initial one-third of the logarithmic phase. In other embodiments, the bacterial host cells will be cultured until they are in the mid-logarithmic phase of growth. Mid-logarithmic phase of growth encompasses the middle one-third of the logarithmic phase of growth. In yet other embodiments, the bacterial host cells will be cultured until they are in the late-logarithmic phase of growth. As used herein, late logarithmic phase of growth will be growth in the final one-third of the logarithmic phase.

The skilled artisan understands that there are many methods of culture that can be employed for the bacterial host cells. For example, the bacterial host cells can be cultured at various temperatures including room temperature, 37 degrees Celsius (hereinafter designated ° C.), 4° C., and all temperatures in-between. The bacterial host cells can be cultured by shaking in some embodiments, while in other embodiments, they will be statically cultured.

Following appropriate culture conditions, bacterial host cells can be harvested. Harvesting cells is the process where bacterial host cells are isolated and then processed in some way to create a solution of viable cells for further use. An appropriate method of harvesting is any method of isolating the bacterial cells which maintains the viability of at least a majority of the cells. A common method of harvesting includes centrifugation. The speed and time of centrifugation for harvest may vary. Harvesting by centrifugation may take place at any speed between 2600×g and 27,000×g. In one or more embodiments, the centrifugal speed will be 16,264×g. In some embodiments, the time of centrifugation will be between 5 minutes and 15 minutes. In certain embodiments, the time of centrifugation will be 10 minutes. Bacterial host cells can be placed on ice prior to harvest. Bacterial host cells may also be refrigerated.

During harvest, washing may take place in any appropriate solution. Bacterial host cells will generally be washed prior to transfer of a nucleic acid of interest into them. In one embodiment, the bacterial host cells will be washed with a MgCl₂ solution. When used as a washing solution, the concentration of MgCl₂ can be in any range between about 1 mM and 100 mM. In some embodiments, the concentration of MgCl₂ will be in the range of about 5 mM to 20 mM. The concentration of MgCl₂ used to wash the cells can be about 10 mM. In an exemplary embodiment, the washing solution is SM buffer. The washing solution can be cooled below room temperature. In an embodiment, the washing solution will be ice cold. Bacterial host cells can be washed more than once. If bacterial host cells are washed more than once, it is to be understood that there is no requirement that the washing solution be the same for each wash. In one embodiment, the bacterial host cells will be washed twice. In other embodiments, the bacterial host cells will be washed more than twice. In many embodiments, the bacterial host cells will be washed until the washing solution is non-ionic. A solution is considered non-ionic when the concentrations of ions is adequately low so that when electricity is discharged into the bacterial host cells, little or no additional current is carried into the bacterial host cells.

Following any wash steps, bacterial host cells can be re-suspended in an appropriate solution. In certain embodiments, the bacterial host cells will be re-suspended in order to concentrate them prior to transfer of a nucleic acid of interest. For example, the bacterial host cells can be re-suspended in a reduced volume of re-suspension solution in order to concentrate them at least 100 fold. In many embodiments, the final concentration of cells is approximately 2.5×10¹⁰ cells per milliliter (ml). In some embodiments, the final concentration of cells will be at least 2.5×10¹⁰ cells per ml. In several embodiments, the re-suspension solution will be SM buffer. Sucrose can be added to the re-suspension solution. The concentration of sucrose in the re-suspension solution can be any concentration between about 0.1M and 1M. In one embodiment, the concentration of sucrose is 952 mM. If SM is used as the re-suspension buffer, the MgCl₂ concentration of the SM buffer may vary. In many embodiments, the concentration of MgCl₂ in the SM re-suspension buffer is between about 1 mM and 100 mM. In one of these embodiments, the MgCl₂ concentration is about 3.5 mM MgCl₂. In yet another embodiment, SM buffer comprises sucrose at a concentration of about 952 mM and MgCl₂ at a concentration of about 3.5 mM.

As used herein, the term “nucleic acid of interest” includes, but is not limited to, nucleic acid sequences that encode functional or non-functional proteins, and fragments of those sequences, polynucleotides, and oligonucleotides. A nucleic acid is a polymer of RNA or DNA that is single- or double-stranded, optionally containing synthetic, non-natural or altered nucleotide bases. A nucleic acid of interest in the form of a polymer of DNA can be comprised of one or more segments of cDNA, genomic DNA, or synthetic DNA. A nucleic acid of interest can be obtained naturally or synthetically, e.g. using PCR or mutagenesis. Further, a nucleic acid of interest can be circular, linear, or supercoiled in its topology. Although not limited to such sizes, certain embodiments employ nucleic acids of interest ranging about from 0.1 kb to 20 kb, depending on factors well known to those of skill in the art.

In one embodiment of the invention, the nucleic acid of interest is a ferulic acid esterase gene. Such a nucleic acid of interest can encode a native or mutant gene for ferulic acid esterase. In another embodiment, the nucleic acid of interest is a mutated ferulic acid esterase gene, such as, for example, a knock-out mutant that is described below.

Transfer of a nucleic acid of interest into the bacterial host cells may include transient transfer or permanent incorporation by either autonomous replication or integration into the genome. In certain embodiments, incorporation of a nucleic acid of interest will result in the knock-out of production of a protein native to the bacterial host cell species. In several embodiments, more than one nucleic acid of interest will be transferred into the bacterial host cells.

A nucleic acid of interest can be a nucleic acid from Lactobacillus buchneri. In certain embodiments, the nucleic acid of interest is nucleic acid from Lactobacillus buchneri strain PTA-6138. The Lactobacillus buchneri nucleic acid of interest can be a gene. A gene refers to a nucleic acid fragment that expresses a specific protein, including regulatory sequences preceding (5′ non-coding sequences) and following (3′ non-coding sequences) the coding sequence. Lactobacillus buchneri genes used as a nucleic acid of interest can be native genes or mutant genes. “Native genes” refer to genes as found in nature. In contrast, “Mutant genes” refer to any gene that is not a native gene, i.e. a gene comprising regulatory and coding sequences that are not found together in nature. Accordingly, a mutant gene may comprise regulatory sequences and coding sequences that are derived from different sources, or regulatory sequences and coding sequences derived from the same source, but arranged in a manner different than that found in nature. Mutant genes also include genes that can be missing regulatory sequences and coding sequences or have additional or altered regulatory sequences and coding sequences.

A nucleic acid of interest can be a native gene or mutant gene for an enzyme. In some embodiments, a nucleic acid of interest will be a gene for an enzyme native to Lactobacillus spp. In certain embodiments, a nucleic acid of interest will be a gene for an enzyme native to Lactobacillus buchneri. A nucleic acid of interest may encode a native or mutant gene for ferulic acid esterase. Ferulic acid esterase is explained in detail in U.S. Patent Application Publication No. 20080115241, incorporated by reference.

In many embodiments, a nucleic acid of interest will be capable of modifying ferulic acid esterase activity in the bacterial host. A “knock-out” or “knocking-out” defines a genetic organism or genetic technique in which a bacterium is engineered to carry inoperative genes. For example, a nucleic acid of interest can be capable of knocking out or inactivating ferulic acid esterase in Lactobacillus buchneri. Knock-out or inactivation may occur either by removing a native ferulic acid esterase gene or by blocking all or some of the ferulic acid esterase activity. An example of a Lactobacillus buchneri ferulic acid esterase knock-out is Lactobacillus buchneri strain LN7113. In contrasting embodiments, a nucleic acid of interest will be capable of increasing ferulic acid esterase activity. An increase in ferulic acid esterase activity can be accomplished by increasing ferulic acid esterase expression or translation efficiency. An increase in ferulic acid esterase activity may also be achieved by adding more ferulic acid esterase protein, i.e. through transformation of ferulic acid esterase nucleic acid, or by blocking or removing compounds known to decrease ferulic acid esterase amounts or activity.

Multiple nucleic acids of interest can be transferred either concurrently or in a step-wise matter. For example, in some embodiments, nucleic acids of interest that first knock-out ferulic acid esterase and then later restore ferulic acid esterase are contemplated.

In several embodiments, a nucleic acid of interest will be contained in a plasmid or vector. The nucleic acids of interest can be introduced into any desired plasmid or vector.

A plasmid is an extra-chromosomal DNA molecule separate from chromosomal DNA. Plasmids, which are generally circular, are capable of replicating independently of chromosomal DNA. Some embodiments include nucleic acid of interest on more than one plasmid. There is no limitation to the number of plasmids containing nucleic acid of interest that can be transferred into bacterial host cells.

One of skill in the art understands that individual plasmids are not limiting. Commercial and research plasmids which can be used to transfer nucleic acids are well-known in the art and have been used by molecular biologists for decades. Any plasmid capable of transferring a nucleic acid of interest into a bacterial host cell can be used. For a review of the use of plasmids in bacterial transformation, see Plasmids: Current Research and Future Trends, Ed. Georg Lipps (2008).

Plasmids used in genetic engineering are also called vectors. Example vectors that can be used with selected embodiments include, but are not limited to, pGK12 and pG+host 5. Other example vectors include but are not limited to pGK12, pNZ12, pK252, pK253, and pAMbetal. pG+host 5 can be used to knock-out or introduce ferulic acid esterase to bacterial host cells through integration/excision mutagenesis. Use of pG+host 5 as a vector is well described in U.S. Pat. Nos. 6,025,190 and 5,919,678, which are hereby incorporated by reference. Vectors used with embodiments may include genes to antibiotic resistance or other selectable markers in order to allow for later selection. An example vector with a selectable marker is a vector that contains the gene conferring erythromycin resistance. Many times, vectors with selectable markers will be transferred into the bacterial host cells either using the same vectors as one containing a nucleic acid of interest or on a different vector. Thus, the nucleic acids of interest can be introduced into a plasmid or vector.

When mixing bacterial host cells with a nucleic acid of interest, the concentration ratio between the nucleic acids and the bacterial host cell may vary. In some embodiments, the concentration of bacterial host cells (e.g., Lactobacillus buchneri cells) to nucleic acid will be between about 4×10⁵ cells per μg nucleic acid and 4×10¹¹ cells per μg nucleic acid. In one embodiment, the concentration of bacterial host cells to the nucleic acid of interest is about 1×10⁹ cells per μg nucleic acid. Unless specifically stated, the concentration ratio of bacterial host cell to nucleic acid commonly includes the concentration of all of the nucleic acid being transferred into the cell, i.e. vector plus nucleic acid of interest.

A method of transferring a nucleic acid of interest into a bacterial host cell includes electroporation. As used herein, electroporation includes any method of using electrical pulses or electrical discharges to increase the permeability of the cell membrane. Electric pulses delivered by electroporation include square wave pulses, exponential wave pulses, unipolar oscillating wave forms of limited duration, bipolar oscillating wave forms of limited duration, and other wave forms that generate electric fields. In an exemplary embodiment, a nucleic acid of interest is transferred into the bacterial host cell using a square wave or an exponential wave electric impulse. Those of skill in the art will know of apparatus' which delivers a desired wave form. An apparatus that can be used is a Bio-Rad Electroporator plus Pulse Controller® (available from Bio-Rad, Hercules, Calif.), which delivers an exponential wave pulse.

Electroporation takes into account three variable electrical parameters: field strength, capacitance, and resistance. In many embodiments, the field strength is about between 0.5 kV/cm and 25 kV/cm. In certain embodiments, the field strength is about 10 kV/cm. In some embodiments, the amount of voltage applied between the electrodes is in the range of about 0.05 kV to 2.5 kV when using an electroporation cuvette with a gap size of 0.1 cm. In one of these embodiments, the amount of voltage applied is 1.0 kV. As the skilled artisan understands, in order to maintain the appropriate field strength, the voltage applied will need to vary with different electroporation cuvette gap sizes. If cuvettes with different gap sizes are used, applied voltage can be altered appropriately. In many embodiments, electroporation will take place in electroporation cuvettes. Electroporation cuvettes are readily available commercially. These cuvettes are well known in the art and come in a variety of sizes and materials. The skilled artisan understands that the cuvettes can be disposable. The cuvette material or the material of the electrode in the electroporation cuvette is not particularly limiting. In many embodiments, the electrode is made from aluminum. In other embodiments, the electrode can be another type of metal. Electroporation cuvettes also come in a variety of electrode gap sizes. The gap size is the fixed value of the inter-electrode distance. In one embodiment, the electroporation cuvette has a gap size of 0.1 cm.

Resistance-capacitance time constants for electroporation can be between about 2.5 milliseconds and 25 milliseconds. In some embodiments, the resistance-capacitance time constant is 8.0 milliseconds. As is well understood in the art, to change the time constant of the pulse, either the capacitance can be adjusted or the resistance can be altered. In many embodiments, the capacitance will be 25 uF. In specific embodiments, resistance values can be either 400 ohms or 1000 ohms. Nevertheless, it is understood that capacitance and resistance values can be altered in any manner to keep a desired time constant.

Following electroporation, the bacterial host cells can be washed. In many cases, the bacterial host cells will be washed out of the electroporation cuvette immediately. The washing solution can be the same or different than the washing solution used prior to electroporation. In one embodiment, the post-electroporation washing solution can be MRS-SM solution containing about 0.5 M Sucrose and 0.1 M MgCl₂. The washing solution can be filtered prior to its use. The washing solution may also be pre-warmed. In an embodiment, the washing solution is pre-warmed to about 30° C.

The bacterial host cells can be allowed to recover following electroporation. Recovery times may vary. For example, the bacterial host cells can be allowed to recover for about 1 hour, 2 hours, 3 hours, 4 hours, or longer (or any time period between). Recovery temperatures may also vary. Applicable recovery temperatures include those about between 25° C. to 40° C. In specific embodiments, the bacterial host cells may recover at 30° C. or 37° C. The bacterial host cells can be shaken or kept static during their recovery period. In one embodiment, the bacterial host cells are allowed to recover for about 4 hours at about 30° C. in a static environment.

Antibiotics can be added to the recovering bacterial host cells if an antibiotic resistance gene has been transferred during electroporation. In certain embodiments, antibiotics will be added to the bacterial host cells in the last hour of recovery. The type of antibiotic, although not particularly limiting, includes erythromycin. In several examples, the concentration of erythromycin will be about between 0.001 μg/ml and 1 μg/ml. In one of these examples, the erythromycin concentration is 0.01 μg/ml.

Bacterial host cells may also be plated following recovery. Any plating medium that allows for bacterial growth can be used. In some embodiments, the plating medium will be a growth medium. An example growth medium includes but is not limited to MRS. The plating medium may optionally include a substance, such as an antibiotic, that allows selection between those bacterial host cells that contain a nucleic acid of interest and those that do not. Types of selection include, but are not limited to, resistance to antibiotics, assimilability of special sugars, and special requirements for amino acids. For example, if a nucleic acid of interest is contained in a vector that confers erythromycin resistance, the plating medium can be MRS plus erythromycin. Selection using chloramphenicol may also be used. Thus, an embodiment where the growth medium is MRS plus chloramphenicol is contemplated.

In one exemplary embodiment, a transformed bacterial host cell with a mutant ferulic acid esterase gene is constructed. A mutant ferulic acid esterase gene is inserted into a pG+host5 vector. The mutant ferulic acid esterase gene is then transferred into bacterial host cells using electroporation. The mutant ferulic acid esterase then becomes part of the bacterial host cells genome by recombination. The bacterial host cells can be Lactobacillus buchneri cells. In some embodiments, the Lactobacillus buchneri cells will be Lactobacillus buchneri strain PTA-6138 cells. The mutant ferulic acid esterase gene can be a knock out. New strains of Lactobacillus buchneri with mutant, including knocked-out ferulic acid esterase, can be used as negative controls. For example, a transformed Lactobacillus buchneri strain can be used in an assay for studying the impact of ferulic acid esterase activity. One example of such an assay would be inoculating whole plant material, such as whole plant silage, with a transformed Lactobacillus buchneri strain. For example, the transformed Lactobacillus buchneri strain could be missing ferulic acid esterase. In other embodiments, the transformed Lactobacillus buchneri strain could have a non-functional ferulic acid esterase. In many embodiments, this whole plant silage will be whole plant corn silage. Whole plant silage treated with a transformed Lactobacillus buchneri strain can then be compared to whole plant silage treated with a native Lactobacillus buchneri strain. As used herein, the term “treated” refers to introduction of viable microbes to plant material or animals. One way, although certainly not limiting, of studying the impact of ferulic acid esterase activity is to measure the difference in digestibility of whole plant material treated with either native or transformed strains of Lactobacillus buchneri. The skilled artisan is well versed in these types of assays. Ferulate esterase nucleic acid and polypeptides as well as ferulate esterase producing strains of bacteria and methods of using them to treat whole plant material are expanded on in U.S. patent application Ser. Nos. 11/939,343 and 11/217,764 and their related continuation applications, all of which are hereby incorporated by reference.

The invention provides Lactobacillus spp. cells comprising a nucleic acid of interest that was transferred into the Lactobacillus spp. cells by the methods of the present invention. In some embodiments, the nucleic acid of interest is a heterologous nucleic acid of interest. As used herein, “heterologous” in reference to a nucleic acid of interest is a nucleic acid that originates from a foreign species, or, if from the same species, is substantially modified from its native form in composition and/or genomic locus by deliberate human intervention. Such a substantial modification in composition comprises at least one difference in a nucleotide in the nucleotide sequence of the heterologous nucleic acid of interest, when compared to the corresponding native nucleic acid of interest. Such a difference can be the addition or deletion of a nucleotide or the substitution of one nucleotide a particular position in the nucleotide sequence with a diffence nucleotide. Such a substitution may or may not result in a change in the amino acid sequence of any protein encoded thereby.

In one embodiment, the invention provides Lactobacillus buchneri cells comprising a nucleic acid of interest that was transferred into the Lactobacillus buchneri cells by the methods of the present invention.

Exemplary Lactobacillus buchneri strains include but are not limited to the following: PTA-2493, PTA-2494, PTA-2495, PTA-6138 and ATCC 202118NRRL B-30986, NRRL B-30987, NRRL B-30988, NRRL B-30989, NRRL B-30990, NRRL B-30991, and NRRL B-30866. A deposit of the following microorganisms has previously been made with the American Type Culture Collection (ATCC), 10801 University Blvd., Manassas, Va. 20110-2209: Lactobacillus buchneri PTA-2493, PTA-2494, PTA-2495, PTA-6138 and ATCC 202118. These microorganisms, deposited with the ATCC on Sep. 21, 2000 (PTA-2493, PTA-2494, PTA-2495), Aug. 3, 2004 (PTA-6138), and Apr. 29, 1998 (ATCC 202118) were taken from the same deposit maintained at Pioneer Hi-Bred International, Inc (Des Moines, Iowa).

The strains Lactobacillus buchneri NRRL B-30986, NRRL B-30987, NRRL B-30988, NRRL B-30989, NRRL B-30990, NRRL B-30991, and NRRL B-30866 were previously deposited on Nov. 16, 2006 (NRRL B-30986, NRRL B-30987, NRRL B-30988, NRRL B-30989, NRRL B-30990, NRRL B-30991) and Aug. 6, 2005 (NRRL B-30866) with the Agricultural Research Service (ARS) Culture Collection, housed in the Microbial Genomics and Bioprocessing Research Unit of the National Center for Agricultural Utilization Research (NCAUR), under the Budapest Treaty provisions. The address of NCAUR is 1815 N. University Street, Peoria, Ill., 61604.

Additionally, it may be advantageous to treat whole plant material with transformed Lactobacillus buchneri strains that express increased levels of ferulate acid esterase. As used herein, the term “plant material” refers to material of plant origin. In many cases, the plant material will be silage. The term “silage” is intended to include all types of fermented agricultural products such as grass silage, alfalfa silage, wheat silage, legume silage, sunflower silage, barley silage, whole plant corn silage (WPCS), sorghum silage, fermented grains and grass mixtures. Transformed Lactobacillus buchneri strains that over-express ferulate esterase activity can be used in assays similar to the one set forth above. Over-expressing Lactobacillus buchneri strains may also be used to treat whole plant material in order to increase its digestibility. The digestibility of the whole plant material can be measured using an in situ neutral detergent fiber digestibility test. These tests are well known in the art.

Animals can be fed plant material treated with Lactobacillus spp. transformed strains. Animals may also be treated with Lactobacillus spp. transformed strains directly. Animals that may benefit from embodiments covering transformed Lactobacillus spp. strains, especially those with increased level of ferulate acid esterase, include mammals and birds, including but not limited to ruminant, equine, bovine, porcine, caprine, ovine and avian species, e.g., poultry.

Embodiments are further defined in the following Example. It should be understood that this Example, while indicating certain embodiments, is given by way of illustration only. From the above discussion and this Example, one skilled in the art can ascertain the essential characteristics current, and without departing from the spirit and scope thereof, can make various disclosed changes and modifications to the embodiments to adapt to various uses and conditions. Thus, various modifications of the embodiments, in addition to those shown and described herein, will be apparent to those skilled in the art from the foregoing description. Such modifications are also intended to fall within the scope of the appended claims.

The following examples are offered by way of illustration and not by way of limitation.

### Example 1

### Electroporation of Lactobacillus buchneri LN4017 (PTA-6138)

Strain LN4017 was grown without shaking at 37° C. for 26 hours in 10 ml De Man Rogosa Sharpe broth (MRS; Difco™ Lactobacilli MRS; Becton Dickinson and Company, Sparks, Md.), prepared as described by the manufacturer. Sub-cultures of 0.1 ml, 0.3 ml, 0.5 ml or 0.8 ml of culture were made into 100 ml filter-sterilized pre-warmed MRS broth+0.5 M sucrose+1% glycine in side arm flasks. The sub-cultures were grown overnight without shaking at 37° C. until an OD₆₀₀ of 0.6 had been reached. The 0.3 ml sub-culture was selected and after a 10 min. incubation on ice, the bacterial cells were harvested by centrifugation (16,264×g; 10 min.) at 4° C. and washed in 100 ml cold 10 mM MgCl₂ to remove extracellular polysaccharides. After re-centrifugation, the cells were washed twice to reduce sample resistance with 100 ml SM (952 mM sucrose, 3.5 mM MgCl₂), with centrifugations between washes. The cells were gently re-suspended to a total volume of 1.0 ml in ice-cold SM.

Plasmid DNA was prepared from the Dam/Dcm methylase negative E. coli strain GM2163 (New England BioLabs, Ipswich, Mass.) using Nucleobond PC columns (Clontech, Mountain View, Calif.). One μg of vector pGK12 (Jan Kok, University of Groningen, The Netherlands) or clone pG+host5-1 (vector pG+host5 (INRA, Paris, France) containing an insert of LN4017 DNA) was mixed with 40 μl of prepared bacterial cells in a microfuge tube on ice. The cell and DNA mixture was transferred to a 0.1 cm cuvette (Bio-Rad, Hercules, Calif.) on ice. Electroporation was performed using a Bio-Rad Gene Pulser® Apparatus with Pulse Controller using a capacitance of 25 μF, a voltage of 1.0 kV and a resistance of 400 or 1000 ohms. The Bio-Rad Gene Pulser® Apparatus produces an exponential decay pulse.

Cells were removed from the cuvette immediately after electroporation by rinsing with 1 ml pre-warmed filter-sterilized MRS-SM (MRS with 0.5 M sucrose, 0.1 M MgCl₂). A cell recovery period was provided by incubation without shaking at 30° C. for 4 hours. Erythromycin resistance was induced in pG+host5-1 by adding Erythromycin to a final concentration of 0.01 μg/ml during the last hour of incubation. Transformants were selected by plating 100 μl cells on MRS+10 μg/ml Chloramphenicol (pGK12) or by plating 50 μA cells on MRS+2 μg/ml Erythromycin (pGK12 or pG+host5-1). Plates were incubated in buckets with BD GasPak™ EZ sachets (Becton Dickinson and Company) at 30° C. for 3 days. Results are given in Table 1. Transformation was confirmed in selected isolates by plasmid profile analysis.

TABLE 1
Transformation efficiency of Lactobacillus buchneri strain LN4017.

| Transforming | Resistance | Time Constant | Antibiotic | Transformants |
|---|---|---|---|---|
| Vector | (ohms) | (milliseconds) | Selection^{a} | per μg DNA |
| pGK12 | 400 | 6.1 | Cm | 1 |
| pGK12 | 400 | 5.9 | Em | 2 |
| pG + host5-1 | 400 | 6.2 | Em | 0 |
| pG + host5-1 | 400 | 6.1 | Em | 1 |
| pGK12 | 1000 | 8.0 | Cm | 4 |
| pGK12 | 1000 | 8.0 | Em | 2 |
| pG + host5-1 | 1000 | 7.6 | Em | 0 |
| pG + host5-1 | 1000 | 8.7 | Em | 0 |

^{a}Cm signifies 10 μg/m1 of Chloramphenicol, Em signifies 2 μg/ml of Erythromycin.

The word “exemplary” is used herein to mean serving as an example, instance, or illustration. Any aspect or design described herein as “exemplary” is not necessarily to be construed as preferred or advantageous over other aspects or designs. Exemplary embodiments may be implemented as a method, apparatus, or article of manufacture.

The article “a” and “an” are used herein to refer to one or more than one (i.e., to at least one) of the grammatical object of the article. By way of example, “an element” means one or more element; reference to “a component” can include a combination of two or more components; reference to “an osmotic stabilizer” can include mixtures of stabilizers, and the like.

All publications and published patent documents cited in this specification are incorporated herein by reference to the same extent as if each individual publication or published patent document was specifically and individually indicated to be incorporated by reference.

Having illustrated and described the principles of the embodiments, it should be apparent to a person skilled in the art that the embodiments can be modified in arrangement and detail without departing from such principles. All modifications that are within the spirit and scope of the appended claims are claimed.

That which is claimed:

1. A method for transferring a nucleic acid of interest into Lactobacillus buchneri cells comprising:
(a) culturing the Lactobacillus buchneri cells in a growth medium plus an agent to weaken the cell wall;
(b) harvesting and washing the Lactobacillus buchneri cells;
(c) mixing the Lactobacillus buchneri cells with at least one nucleic acid of interest in a concentration of 4×10⁵ cells per μg nucleic acid to 4×10¹¹ cells per μg nucleic acid; and
(d) subjecting the Lactobacillus buchneri cells plus nucleic acid of interest to electroporation, thereby permitting the transfer of the nucleic acid of interest into the Lactobacillus buchneri cells, wherein the electroporation uses a field strength of between about 0.5 kV/cm and about 25 kV/cm and a time constant of between about 2.5 milliseconds and about 25 milliseconds.

2. The method of claim 1 further comprising: adding at least one osmotic stabilizer to the growth medium.

3. The method of claim 2, wherein at least one osmotic stabilizer is sucrose.

4. The method of claim 1, wherein the agent to weaken the cell wall is glycine in a concentration between about 0.1% and about 5%.

5. The method of claim 1, wherein the growth medium is MRS broth.

6. The method of claim 1 further comprising culturing the Lactobacillus buchneri cells until the Lactobacillus buchneri cell culture is in logarithmic growth phase.

7. The method of claim 1, wherein the Lactobacillus buchneri cells are washed with a MgCl₂ solution.

8. The method of claim 1, wherein the Lactobacillus buchneri cells are washed until the washing solution is low-ionic.

9. The method of claim 1 further comprising re-suspending the Lactobacillus buchneri cells following washing to a concentration of between about 1×10⁸ cells/ml and about 1×10¹² cells/ml.

10. The method of claim 9, wherein the Lactobacillus buchneri cells are re-suspended in SM buffer.

11. The method of claim 1, wherein the concentration ratio of Lactobacillus buchneri cells to a total amount of nucleic acid to be transferred is between about 4×10⁵ cells per μg nucleic acid and 4×10¹¹ cells per μg nucleic acid.

12. The method of claim 1, wherein the electroporation uses an exponential decay pulse.

13. The method of claim 1, wherein the resistance-capacitance time constant is about 8.0 milliseconds.

14. The method of claim 1 further comprising rinsing the Lactobacillus buchneri cells from the cuvette following electroporation.

15. The method of claim 1 further comprising allowing the Lactobacillus buchneri cells to recover for a period following electroporation.

16. The method of claim 15, wherein the period is between about 1 hour and about 8 hours.

17. The method of claim 15, wherein the cells recover at a temperature between about 25° C. and about 40° C.

18. The method of claim 15, further comprising adding an antibiotic to the Lactobacillus buchneri cells during the period of recovery.

19. The method of claim 18 further comprising plating the Lactobacillus buchneri cells on a growth medium following recovery.

20. A method for transferring nucleic acids of interest into Lactobacillus buchneri cells comprising:
(a) culturing Lactobacillus buchneri cells at 37° C. in MRS broth plus 0.5M sucrose and 1% glycine until the Lactobacillus buchneri cell culture reaches mid-logarithmic growth phase;
(b) incubating the Lactobacillus buchneri cells on ice for 10 minutes;
(c) harvesting the Lactobacillus buchneri cells by centrifugation;
(d) washing the Lactobacillus buchneri cells in cold 10 mM MgCl²;
(e) washing the cells twice in SM;
(f) re-suspending the cells in cold SM to a concentration of 2.5×10¹⁰ cells/ml;
(g) mixing the cells with a nucleic acid of interest in a concentration ratio of about 1×109 Lactobacillus buchneri cells per μg nucleic acid;
(h) subjecting the Lactobacillus buchneri cells plus nucleic acid of interest to electroporation at a field strength of between about 0.5 kV/cm and about 25 kV/cm and a time constant of between about 2.5 milliseconds and about 25 milliseconds, thereby permitting the transfer of the nucleic acid of interest into the Lactobacillus buchneri cells;
(i) washing the Lactobacillus buchneri cells from the cuvette following electroporation with MRS broth with 0.5 M sucrose and 0.1 M MgCl₂;
(j) allowing the Lactobacillus buchneri cells to recover for a 4 hour period at 30° C.; and
(k) inducing erythromycin resistance by adding erythromycin to a final concentration of 0.01 μg/ml during the last hour of recovery.

21. A method of constructing a Lactobacillus buchneri transformant comprising subjecting Lactobacillus buchneri cells plus a nucleic acid of interest to electroporation, thereby permitting the transfer of the nucleic acid of interest into the Lactobacillus buchneri cells, wherein the electroporation uses a field strength of between about 0.5 kV/cm and about 25 V/cm and a time constant of between about 2.5 milliseconds and about 25 milliseconds.

22. The method of claim 21, wherein the nucleic acid of interest is a ferulic acid esterase gene.

23. The method of claim 21 further comprising introducing the nucleic acid of interest into a Lactobacillus buchneri genome by recombination.
